(19) United States
(12) Patent Application Publication
(10) Pub. No.: US 2004/0199082 A1
(43) Pub. Date: Oct. 7, 2004

(54) Selctable notch filter circuits

(76) Inventors: Alan H. Ostroff, San Clemente, CA (US); James William Phillips, Fountain Valley, CA (US)

Correspondence Address: Daniel M. Chambers, Biotechnology Law Group, 658 Marsolan Avenue, Solana Beach, CA, 92075-1931, US

(21) Appl. No.: 10/406,454

(22) Filed: Apr. 3, 2003

(51) Int. Cl. A61B 5/04

(52) U.S. Cl. 600/509

(57) ABSTRACT

Described are selectable notch filter circuits comprising at least two different notch filter capabilities, each of which is capable of filtering interference of a designated fundamental frequency and a second harmonic of the designated fundamental frequency from an electrical signal. Each of the notch filters of the circuit is specific for real time filtering of a different designated fundamental frequency and a second harmonic thereof from digitized signal data input into the circuit. The filtering capability of each filter is dictated by control logic, which uses a coefficient set specific for the designated fundamental frequency and harmonics thereof. By using different coefficient sets, different designated fundamental frequencies and at least their second harmonic frequencies can be filtered from digitized signal data input into the circuit. Because the control logic can utilize at a given time any one (or, if desired, none) of the coefficient sets available to it, different interfering fundamental frequencies can be filtered, if and as necessary, from digitized input signal data collected over time at a substantially equivalent sampling rate. Also described are devices including one or more such selectable notch filter circuits, including implantable medical devices such as implantable cardioverter/defibrillators, as well as methods of using such devices.

### TECHNICAL FIELD

This invention relates to articles, machines, and processes useful in removing interference from electrical signals. More particularly, the invention concerns circuits that employ a plurality of digital notch filters at least one of which may be selected and used to filter interference present in an electrical signal within a machine, for example, an implantable medical device.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

Many microelectronic devices are sensitive to various types of electromagnetic interference, or “noise.” Noise refers to the unintentional or unwanted introduction of energy of a specific frequency, or range of frequencies, into an electrical signal. A predominant cause of noise in electrical signals in microelectronic devices is so-called “line frequency noise.” Line frequency noise (and noise stemming from its harmonic frequencies, which occur at integer multiples of the underlying line frequency) stems from the near ubiquitous presence of generated electricity and devices powered by electricity. In certain parts of the world, for example, the United States, electrical energy is distributed as an alternating current that cycles at 60 Hz, which can cause noise at 60 Hz and harmonics thereof. Similarly, in many other parts of the world, notably in Europe and in many parts of Asia, electricity is distributed as an alternating current that cycles at 50 Hz. These line frequencies can thus cause noise in microelectronic devices at 50 Hz and 60 Hz and their harmonic frequencies. In addition to line noise, other sources of noise include microwave generators, metal detectors, theft-prevention systems, and medical imaging devices, among others. It is also important to consider that frequently, more than one source of noise may be encountered at a given time. As such, an electrical signal may contain many different components attributable to noise of different fundamental frequencies.

Because noise introduces unwanted components into an electrical signal, it can create significant problems, particularly in the context of implantable medical devices such as pace markers and other devices that monitor and provide therapeutic electrical stimuli to the heart, as well as other devices that employ signals comprised of very small currents during normal operation. For example, noise may drown out a signal generated by a sensor that indicates the onset of an abnormal condition such as an irregular heartbeat. Unless that part of the signal attributable to the noise is filtered out, the device may not recognize the onset of the abnormal condition, potentially to the great detriment of the patient in whom the device is implanted. While a number of approaches have been developed to address the problem of noise in the signals of microelectronic devices, there remains a pressing need for effective solutions.

### SUMMARY OF THE INVENTION

It is an object of this invention to provide a circuit that can be used in a device for analyzing the data content of an electrical signal initiated by a sensor, wherein the circuit comprises at least two different notch filters each of which is capable of filtering interference of a designated fundamental frequency and a second harmonic of the designated fundamental frequency from the electrical signal. Broadly, a “filter” is any device element that separates one frequency, or a band of frequencies, from an input spectrum. A “notch” filter is a filter for rejecting (or removing or filtering) a specific frequency, or range of frequencies, from an input signal.

Thus, in one aspect, the invention concerns circuits comprised of hardware configured to serve at a given time as a selected one of a plurality of notch filters. Various configurations can be used to accomplish this end. For example, some embodiments will involve a circuit configured to receive and implement different sets of coefficients, each of which is specific for filtering in real time a different frequency, or small range of frequencies, from an input signal. In other embodiments, a plurality of circuits is present, each of which is configured to filter in real time a different frequency, or small range of frequencies, from an input signal. Regardless of configuration, such “notch filtering” may be performed continuously or, preferably, only when “noise” or other interference is detected in the input signal.

In the embodiments of this aspect, each of the notch filters is specific for real time filtering of a different designated fundamental frequency and a second harmonic thereof from digitized signal data input into the circuit. The filtering capability of each filter is dictated by control logic that uses a coefficient set specific for the designated fundamental frequency and harmonics thereof of the particular filter. In embodiments employing a single circuit, the coefficient set to be implemented may be dictated by the presence of interference of a predetermined fundamental frequency in the input signal. A coefficient set corresponding to that frequency may then be retrieved from a memory and implemented by the control logic to effect the desired filtering. In other embodiments, for example, those where multiple circuits each specific for a different fundamental frequency are present, selection and retrieval of coefficient sets is not required. Instead, the circuit designed to filter the “noise” of the particular frequency (or small range of frequencies) may use, or its output selected, for further use. Again, by using different coefficient sets, different designated fundamental frequencies and at least their second harmonic frequencies can be filtered from digitized signal data input into the circuit. Through the use of circuits according to the invention, at any given time different interfering fundamental frequencies (and at least their second harmonics) can be filtered, if and as necessary, from digitized input signal data collected in real time, preferably at a substantially equivalent sampling rate.

As described above, in certain embodiments, the circuit comprises at least two notch filters, each specific for real time filtering of a different designated fundamental frequency and at least the second harmonic of the designated fundamental frequency. In other embodiments, the circuit may contain 3 or more notch filters each specific for a different designated fundamental frequency and a second harmonic thereof. As with other embodiments, what differentiates individual notch filters each specific for the same fundamental frequency and a second harmonic thereof is the coefficient set implemented by the control logic.

As will be appreciated, the number and diversity of notch filters in a given circuit are left to the discretion of the circuit designer, but will be dictated in large part by the different fundamental frequencies that may need to be filtered over time from a digitized signal in the device in which the notch filter circuit(s) is(are) deployed. Corresponding to the frequencies to be filtered are the coefficient sets to be used, either in the same circuit or in dedicated filter circuits each specific for a designated fundamental frequency. As will be appreciated, one or more of the coefficient sets may be stored in the circuit itself or, alternatively, one or more of them may be accessed from a memory separate from, but operably connected with, the circuit, for instance, in a look-up table stored in an associated memory.

Certain preferred embodiments employ a circuit wherein the control logic is embedded in the circuit hardware, while in other embodiments, the control logic is provided as software stored in a memory and executed by a processor. Of course, combinations of control logic, some embedded, some software, may also be employed. To achieve maximum energy efficiency, however, most preferred are circuits that contain control logic embedded therein. This control logic can, at a given time, utilize any one of the coefficient sets available to it. Multiple circuits according to the invention may also be produced, such that a device contains two such two or more of such circuits.

It is further preferred that the control logic be designed to be applied to data that is sampled at a substantially constant, or equivalent, rate over time. Sampling rates should approximately be greater than twice, preferably greater than about four or more times, the highest fundamental frequency to be filtered. Useful sampling rates will typically be in the range of from about 240 Hz to about 10,000 Hz, with sampling higher rates being possible; however, slower sampling rates are presently preferred due to energy consumption considerations. Given this, sampling rates of about 256 Hz, 512 Hz, 1024 Hz, 2048 Hz, and 4096 Hz are preferred, with a sampling rate of about 256 Hz being particularly preferred for use in accordance with circuits of the invention. In certain embodiments, over time the sampling rate may be varied, if desired. In such embodiments, the control logic and coefficient sets will be different. Accordingly, a plurality of circuits according to the invention will be available, each of which being specific for the sampling rate then being employed.

This aspect includes embodiments where the electrical signal initiated from the sensor is always passed through the circuit containing the notch filter(s). As will be appreciated, however, the circuit may be bypassed, or the circuit itself may contain a bypass capability. Thus, filtering according to the invention may be continuous or intermittent. Such embodiments include those wherein the circuit of the invention itself includes a bypass, thereby allowing data input into the circuit to avoid filtering, as may be desired to conserve power, when a device employing the circuit is not exposed, or expected to be exposed, to external electrical interference at a given time, etc.

The capability to switch between, or select an output signal from, any of a plurality of notch filters of the circuit at a given time, or to employ a bypass to allow a signal input into the circuit to not be filtered, is provided by a selector. The selector may be external to the circuit, although a selector internal to the circuit is preferred. When the selector is internal, the circuit preferably contains a single input channel. The selector is used to determine which, if any, of the plurality of notch filters of the circuits, or the outputs thereof, is to be implemented. The selector can be controlled by any suitable control logic that, for example, allows the control logic of the circuit to implement one, or none (if a signal bypass is included in the circuit), of the coefficient sets accessible to the circuit. Preferably, the selector responds to a noise detection circuit that determines whether external electrical interference is present and, if so, at which frequency(ies). A suitable notch filter (or multiple notch filters, if multiple circuit are present) can then be selected and implemented by the circuit(s).

In preferred embodiments of this aspect of the invention, the circuit will have the ability to filter at least two different fundamental frequencies (and the second harmonic frequencies of each of them) commonly present as electrical interference in electrical signals in electronic circuits, namely 50 Hz noise and 60 Hz noise caused by the alternating current of the electricity available in much of the world. Which, if any, of these two fundamental frequencies is filtered at a given time is controlled by a selector. In particularly preferred embodiments, the sampling frequency is 256 Hz for signals input into the circuit. In these embodiments, control logic embedded in the circuit implements the 50 Hz notch filter and the 60 Hz filter by implementing the following transfer function:

$$H ( z ) = \frac{( A - B \cdot {z}^{- 1} + A \cdot {z}^{- 2} ) \cdot ( C + D \cdot {z}^{- 1} + C \cdot {z}^{- 2} )}{1 - E \cdot {z}^{- 1} + F \cdot {z}^{- 2}} \qquad Eq . ( 1 )$$

To filter 50 Hz noise, preferred values for coefficients A-F are selected from among the following ranges:

| coefficient | minimum value | maximum value |
|---|---|---|
| A | about 874/1024 | about 966/1024 |
| B | about 600/1024 | about 664/1024 |
| c | about 274/1024 | about 302/1024 |
| D | about 426/1024 | about 472/1024 |
| E | about 548/1024 | about 604/1024 |
| F | about 730/1024 | about 806/1024 |

Particularly preferred coefficients for filtering 50 Hz noise from digitized input signal data input into the circuit are: coefficient A equal to about 115/128 or a decimal representation thereof; coefficient B equal to about 79/128 or a decimal representation thereof, coefficient C equal to about 9/32 or a decimal representation thereof; coefficient D equal to about 7/16 or a decimal representation thereof; coefficient E equal to about 9/16 or a decimal representation thereof; and coefficient F equal to about ¾ or a decimal representation thereof.

Filtering 60 Hz noise from digitized signal data input into the circuit in accordance with the transfer function of Eq. (1), above, is preferably accomplished using a coefficient set wherein the values of coefficients A-F are selected from among the following ranges:

| coefficient | minimum value | maximum value |
|---|---|---|
| A | about 852/1024 | about 940/1024 |
| B | about 159/1024 | about 177/1024 |
| C | about 244/1024 | about 268/1024 |
| D | about 472/1024 | about 520/1024 |
| E | about 182/1024 | about 202/1024 |
| F | about 730/1024 | about 806/1024 |

A particularly preferred coefficient set for filtering 60 Hz interference from digitized input signal data input into a circuit capable of implementing the transfer function of Eq. (1) is as follows: coefficient A is about ⅞ or a decimal representation thereof; coefficient B is about 21/128 or a decimal representation thereof; coefficient C is about ¼ or a decimal representation thereof; coefficient D is about 31/64 or a decimal representation thereof; coefficient E is about 3/16 or a decimal representation thereof; and coefficient F is about ¾ or a decimal representation thereof.

While it is preferred that the values of coefficients in a coefficient set used in implementing a transfer function for a particular filter each be a fraction, preferably a fraction the denominator of which is a factor of two (or a decimal representation thereof), one or more of such coefficients may also be values that require floating point calculations to be made.

Another aspect of the invention relates to devices that contain circuitry operably associated with a selectable notch filter circuit according to the invention. Such devices include implantable medical devices. Preferred embodiments of such devices include those used to monitor and/or administer therapy to the heart of a patient in which the device is implanted. Representative examples of such devices include implantable pacemakers, defibrillators, and cardioverter-defibrillators (ICDs), including those implanted subcutaneously.

When incorporated into a device such as an implantable medical device, a circuit according to the invention will be operatively connected with circuitry for sensing a physiological parameter (e.g., electrical output of the heart, nerve conduction, concentration of one or analytes in a bodily fluid or tissue, etc.) of a patient by analysis of a digitized electrical signal generated by a sensor capable of sensing the physiological parameter. Representative sensors include electrodes, including those placed in direct physical contact with a tissue or organ to be monitored as well as those that do not make physical contact with the monitored tissue or organ. Sensors include analog and digital sensors. When one or more analog sensors is employed, the electrical signal generated by the sensor in the course of monitoring the physiological parameter is preferably converted to a digital signal, for example, by any suitable analog to digital converter, prior to filtering and analysis. As will be appreciated, the purpose of monitoring the physiological parameter is to detect abnormalities. For example, when monitoring the electrical output of a patient's heart, an abnormal condition (including its onset, duration, cessation, effects, etc.) can be detected in various ways. For example, abnormal heart rhythms (called “arrhythmias”) can be detected by measuring heart rate. Arrhythmias include bradycardia, or an abnormally slow heart rate, as well as tacharrhythmias (abnormally rapid heart rates), such as tachycardia and fibrillation. Other heart pathologies can also be monitored, for example, by analyzing the morphology of the electrical waveform being emitted by the heart.

Preferred medical devices for monitoring patients' hearts typically comprise a heart-specific sensing system. Such sensing systems include at least one electrode for sensing electrical signals within a patient's body, specifically, electrical activity from the patient's heart. The electrode(s) may be in direct physical contact with the heart or, alternatively, they may be non-contact electrodes positioned such that a gap exists between the outer surface of the electrode and the heart, although bridging the gap will be a conductor or combination of different conductors (e.g., tissue, other than cardiac tissue, fluid, etc.). Electrodes are typically analog electrodes.

Electrical signals from the heart that are sensed by the electrode are converted to digital form, i.e., the analog signals are digitized. Any suitable analog to digital (A/D) converter can be used for this purpose. The A/D converter may be integrated into the electrode itself, be disposed between the electrode and monitoring circuitry, or be included in the monitoring circuitry. After the electrical signal is digitized, it is then passed through a selectable notch filter circuit according to the invention. Depending upon whether a bypass is included within, or provided before, the selectable notch filter circuit, the digitized signal input into the circuit may be filtered to remove a designated fundamental frequency and at least its second harmonic. After passing through the selectable notch filter circuit, the signal is analyzed by the monitoring circuitry (i.e., the detector) to determine if a heart-specific component is present and, if so, whether the signal is indicative of a heart abnormality (e.g., an abnormal heart rhythm). In certain preferred embodiments, the detector is an R-wave detector. Data collected by the device may be stored for later retrieval and analysis, transmitted to a distal location (e.g., a base station for re-transmission to a data collection center, to a doctor or hospital, etc.), or used to initiate a therapy in the event an abnormal condition is detected.

Implantable medical devices for monitoring and treating abnormal cardiac conditions are well suited for application of the instant selectable notch filter circuits. Representative examples include ICDs, which not only monitor the heart, but also enable electrical therapy (e.g., defibrillation and/or cardioversion) to be delivered to the heart immediately upon detection (or sensing) of an abnormal heart rhythm.

Other aspects of the invention relate to various methods. For example, the instant selectable notch filter circuits can be used to filter externally generated noise from a digitized electrical signal in an implantable medical device. Briefly, such methods are accomplished by passing a digitized electrical signal through a selectable notch filter circuit according to the invention. A designated fundamental frequency (e.g., 60 Hz or 50 Hz noise) and at least the second harmonic thereof, if present, corresponding to the particular notch filter therefor can then be removed from the digitized electrical signal.

In some embodiments, it is preferred to establish a default setting for filtering a particular fundamental frequency and at least its second harmonic. Here, a selectable notch filter circuit according to the invention is configured such that a specific one of the plurality of notch filters of the circuit is automatically employed until such time as a different interfering frequency (or no such frequency) is detected in the digitized electrical signal. Upon detection of noise having a fundamental frequency different from that filtered by the default notch filter, a different notch filter may be selected from among those others within the circuit's plurality of notch filters. Because filtering utilizes energy, preferably it is performed intermittently.

Another aspect of the invention relates to methods for sensing a physiological parameter of a patient by using an implantable medical device that includes a selectable notch filter circuit according to the invention. A related aspect concerns methods for delivering therapy to a patient, wherein the therapy is administered by an implantable medical device that includes a selectable notch filter circuit according to the invention.

### BRIEF DESCRIPTION OF THE DRAWINGS

These and other aspects and embodiments of the present invention will become evident upon reference to the following detailed description and attached drawings that represent certain preferred embodiments of the invention, which drawings can be summarized as follows:

FIG. 1 is a Z-plane plot for a particularly preferred 50 Hz filter implementing the transfer function of Eq. (1), above.

FIG. 2 shows two frequency response curves, (A) and (B), for a particularly preferred 50 Hz filter implementing the transfer function of Eq. (1), above. Curve (A) plots the magnitude of the response versus normalized frequency. Curve (B) plots the phase of the response versus the normalized frequency.

FIG. 3 is a Z-plane plot for a particularly preferred 60 Hz filter implementing the transfer function of Eq. (1), above.

FIG. 4 shows two frequency response curves, (A) and (B), for a particularly preferred 60 Hz filter implementing the transfer function of Eq. (1), above. Curve (A) plots the magnitude of the response versus normalized frequency. Curve (B) plots the phase of the response versus the normalized frequency.

FIG. 5 represents a prototypic surface electrocardiogram (“ECG”) for a single heartbeat from a human heart. “P”, “Q”, “R”, “S”, and “T” represent different phases of the heartbeat.

FIG. 6 is a flowchart illustrating representative decision processes in a device employing a selectable notch filter circuit according to the invention. In part (a), the decision tree specifies that the circuit initially filter 60 Hz noise but can switch to filtering 50 Hz noise if 50 Hz noise is detected after filtering the input signal with a 60 Hz notch filter. Part (b) represents an initial “no-filter”, or bypass, function, that may included before the functionality represented in part (a). As depicted, no notch filtering is performed unless and until noise is detected.

FIG. 7(a) illustrates a general schematic for a selectable notch filter circuit according to the invention in which either of two notch filter capabilities (60 Hz notch filtering (702) and 50 Hz notch filtering (704)) or filter bypass function (706) can be selected at a given time. Whether filter (702), filter (704), or filter bypass (706) is to be applied at a given time is determined by the selector (708). After filtering, if any, the input signal passes out of the selectable notch filter circuit for analysis by wave detector (710). FIG. 7(b) illustrates a general schematic for a single notch filter circuit (750) into which different coefficient sets can be loaded.

FIG. 8 illustrates a general schematic for an ECG sensing circuit. A signal is initiated from electrode (802). The signal then passes through analog-to-digital converter (804). The digitized input signal is then passed through selectable notch filter circuit (806). As represented, selectable notch filter circuit (806) is upstream of R-wave detector (808) that analyses the contents of the input signal for the presence of data indicative of an abnormal heart condition. Here, selectable notch filter circuit (806) is shown as having two filtering capabilities, namely filtration of either 60 Hz or 50 Hz noise at a given time.

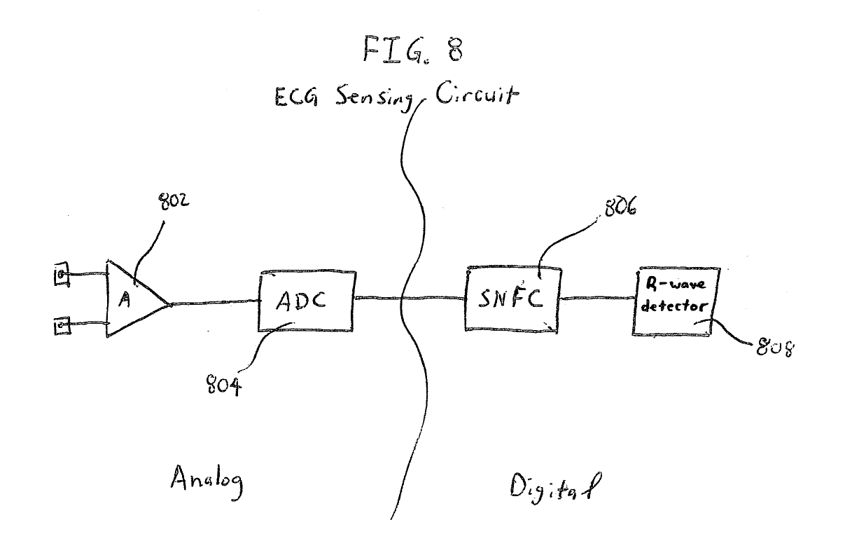

As those in the art will appreciate, the embodiments represented in the attached drawings are representative only and do not depict the actual scope of the invention. For example, the various components of a selectable notch filter circuit may be arranged differently or include additional and/or different components. Moreover, while the following description is in terms of circuitry (digital logic), software versions of this circuitry may be implemented on a general purpose or special purpose processor of the type well known in the art, wherein the software is a computer program that configures circuits in, e.g., a microprocessor, to carry out the filter functions. Thus, the present invention may be implemented in circuitry or in software, or as a combination of circuitry and software, and one of ordinary skill in the art would be able to write such a computer program for carrying out the functions of the filter circuits of the invention in light of this disclosure.

### DETAILED DESCRIPTION OF THE INVENTION

Before the present invention is described in detail, it is understood that the invention is not limited to the particular circuits, configurations, and methodology described, as these may vary. It is also to be understood that the terminology used herein is for the purpose of describing particular embodiments only, and is not intended to limit the scope of the invention defined by the appended claims.

1. Introduction.

The present invention concerns novel, non-obvious selectable notch filter circuits designed to filter interference of a designated fundamental frequency and a second harmonic thereof from a digitized electrical signal input into such a circuit, as well as related devices and methods. Because interference of different fundamental frequencies (and hence harmonics thereof) may be encountered at different times, the circuit is adaptable, or selectable, for at least two different interfering fundamental frequencies that may be encountered by a device containing one or more of these circuits. This adaptability, or selectability, is provided through the use of circuit architecture that enables implementation, if at all, of one of a plurality of different filter transfer functions, each corresponding to a different fundamental frequency (and its second harmonic), depending upon the interfering frequency(ies) detected in the electrical signal input into the circuit. For example, it is expected that devices that employ circuits according to the invention, e.g., ICDs, may be used in different countries. If an ICD patient who lives in the United States travels to Europe, for example, at different times s/he will potentially be exposed to line noise of two different fundamental frequencies: 60 Hz in the U.S.; and 50 Hz in Europe. Accordingly, the device should have the capability to filter each of these different interfering frequencies (and at least their second harmonic frequencies) from electrical signals in the device, as well as the capability to distinguish between interference of different frequencies. Because devices such as ICDs are surgically implanted and have only a limited, non-rechargeable battery-provided energy supply, filtering preferably occurs only when needed, and then only to remove the interfering frequency(ies). Additionally, some types of noise are not specific to a particular frequency, and may not originate from any one source. Hence, it can be difficult to separate such noise from desired, data-containing components of an input signal. A solution to this problem is to provide one or more selectable notch filter circuits according to the invention having multiple filters for different fundamental noise frequencies and second harmonics thereof that may be encountered in the environment where a device incorporating such a circuit is used.

2. The Circuit.

As described above, this invention concerns selectable notch filter circuits preferably comprised of hardware configured to serve at a given time as a selected one of a plurality of notch filters, each of which is specific for real time filtering of a different designated fundamental frequency and a second harmonic thereof from digitized signal data input into the circuit. In each embodiment, the circuit comprises at least two notch filters, or hardware that can implement coefficient sets for each of at least two different notch filters. Thus, depending on the design of the circuit, at given time it will have the capability to implement one of 2-10 or more notch filters each specific for a different designated fundamental frequency and a second harmonic thereof.

The filtering capability of each filter provided by the circuit is dictated by control logic that uses a coefficient set specific for the designated fundamental frequency and harmonics thereof of the particular filter. By using different coefficient sets, different designated fundamental frequencies and at least their second harmonic frequencies can be filtered from digitized signal data input into the circuit. Because a selectable notch filter circuit according to the invention employs the same control logic to implement different coefficient sets for a given transfer function, hence implementing different notch filter functions, by accessing different coefficient sets as needed, it is important that the digitized electronic data input into the circuit as a digitized input signal be collected over time at a substantially equivalent sampling rate. Here, “substantially equivalent sampling rate” means that the data that is sampled for analysis is sampled at a substantially constant, or equivalent, rate over time. In other words, the rate does not vary more than about 10%, preferably not more than about 5%, even more preferably less than about 1%, over time. Any useful sampling rate may be used, although at present, given issues related to processor speed, circuit design, energy consumption, heat dissipation, and the like, useful sampling rates typically range from about 240 Hz to about 10,000 Hz, although higher sampling rates are possible, especially in the future. That being said, slower sampling rates are presently preferred due to the considerations previously mentioned. As such, sampling rates of about 256 Hz, 512 Hz, 1024 Hz, 2048 Hz, and 4096 Hz are preferred, with a sampling rate of about 256 Hz being particularly preferred. Other criteria that may be useful in selecting a data sampling rate include a requirement that the selected sampling rate be at least about twice, and preferably about four times, the highest frequency to be filtered. Thus, if one of the designated fundamental frequencies of a selectable notch filter circuit is 60 Hz, its second harmonic will have a frequency of about 120 Hz, meaning that the minimum sampling rate should be about 240 Hz. As a sampling rate of 256 Hz is the next-highest sampling rate that is a factor of two, that sampling rate would be preferred, provided that 60 Hz is the highest frequency of the different fundamental frequencies that can be filtered by a particular selectable notch filter circuit of the invention.

It will also be appreciated that the invention includes embodiments where the electrical signal initiated from the sensor is always passed through a selectable notch filter circuit and filtered, regardless of whether noise actually contaminates the input signal. Similarly, the invention also includes embodiments where filtering is not performed because no interference (or at least interference that might be filtered in accordance with the invention) is detected in the input signal. This may be accomplished in various ways. For example, the circuit may be bypassed or the circuit itself may contain a bypass, or no-filtering, function. As such embodiments suggest, filtering according to the invention may be continuous or intermittent. Here, “continuous” refers to uninterrupted filtering of a digitized electrical input signal by a notch filter of a selectable notch filter circuit, while “intermittent” refers filtering that is not continuous, as may occur to conserve energy or when no noise is detected, or is expected to be detected, in the input signal. Even so, it will be appreciated that these terms are relative and context-dependent.

Bypassing or directing signal filtration is governed by a selector, which may be external to, but preferably is internal to, the selectable notch filter circuit. For instance, when the selector is internal to the circuit, the circuit will preferably contain a single input channel, and selector determines which, if any, of the outputs of the circuit's plurality of notch filters will be used. The selector can be controlled by any suitable control logic that allows the control logic of the circuit to implement one, or none (if a signal bypass is included in the circuit), of the coefficient sets accessible to the circuit to then be employed. Preferably, the selector is controlled by to a noise detection circuit that determines whether noise is present in the input signal and, if so, at which frequency(ies). A suitable notch filter (or multiple notch filters, if multiple selectable notch filter circuits are provided) can then be selected and implemented.

Below, transfer functions are described for 60 Hz and 50 Hz notch filters, as well as parameters for developing different transfer functions that can be implemented as notch filters. Also described are noise detection methods and circuits, as well as selectors for selecting which, if any, one of the plurality of notch filters in a given selectable notch filter circuit to deploy at a given time.

a. Transfer Functions.

The fundamental frequency (and its harmonics) filtered by a particular notch filter (each a different “designated fundamental frequency”) of a selectable notch filter circuit is dictated by the particular coefficient set then used by the control logic. Thus, the ability to select a coefficient set that corresponds to electrical interference of a designated fundamental frequency from a plurality, or set, of different coefficient sets allows different designated fundamental frequencies (and at least their second harmonic frequencies) to be filtered from digitized signal data input into the circuit. Of course, the invention also envisions embodiments having multiple notch filters, each specific for a different fundamental frequency. In such embodiments, in each filter a different coefficient set is employed, thereby rendering each filter specific for a different fundamental frequency (and at least the second harmonic thereof).

The different coefficient sets used in practicing the invention depend on the transfer function being implemented by the selectable notch filter circuit. Any transfer function that can be implemented as a notch filter to remove a specific fundamental frequency, or range of frequencies (and at least the second harmonic thereof), from a digitized input signal can be used in the practice of the invention. If it is desirable to implement more than one transfer function, it is preferred that a different selectable notch filter circuit be used for each transfer function. In such embodiments, which, if any, transfer function will be implemented at a given time should be selectable by a controller in the operational circuitry of the device. Preferably, such a controller will, for example, issue a command directing operation of a selectable notch filter circuit that implements the desired transfer function.

A particularly preferred transfer function for use in the context of the present invention corresponds to a fourth order infinite impulse response (4^{th} order IIR) filter for filtering line noise, i.e., electrical interference due to the frequency of alternating current used by electrical machinery and appliances in the particular locale. In much of the world, line noise can be detected in electronic devices at a fundamental frequency of 50 Hz or 60 Hz. The second harmonics of these fundamental frequencies are 100 Hz and 120 Hz, respectively. To filter such noise from electronic signals in devices such as implantable medical devices, a 4^{th} order IIR filter was designed to implement the transfer function of Eq. (1), above.

Coefficients A-F for Eq. (1) were derived empirically and independently for a 50 Hz notch filter and a 60 Hz notch filter. In each case, the transfer function of Eq. (1) was implemented as two cascaded filters, one IIR filter and one FIR (finite impulse response) filter. In these embodiments, the sampling frequency was 256 Hz for data input into the circuit. To filter 50 Hz noise, preferred values for coefficients A-F were in the following ranges:

| coefficient | minimum value | maximum value |
|---|---|---|
| A | about 874/1024 | about 966/1024 |
| B | about 600/1024 | about 664/1024 |
| C | about 274/1024 | about 302/1024 |
| D | about 426/1024 | about 472/1024 |
| E | about 548/1024 | about 604/1024 |
| F | about 730/1024 | about 806/1024 |

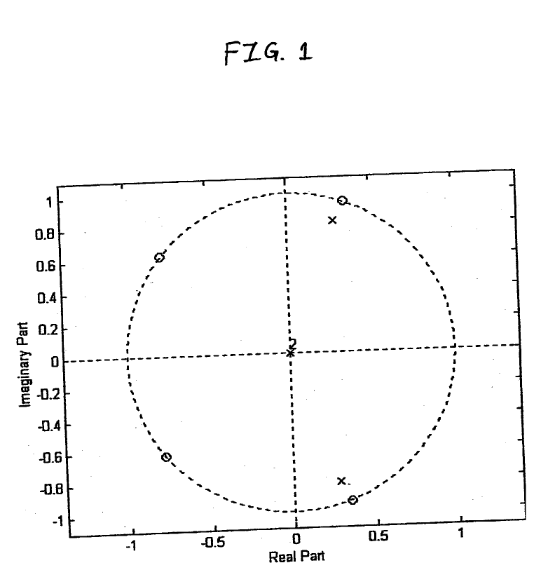

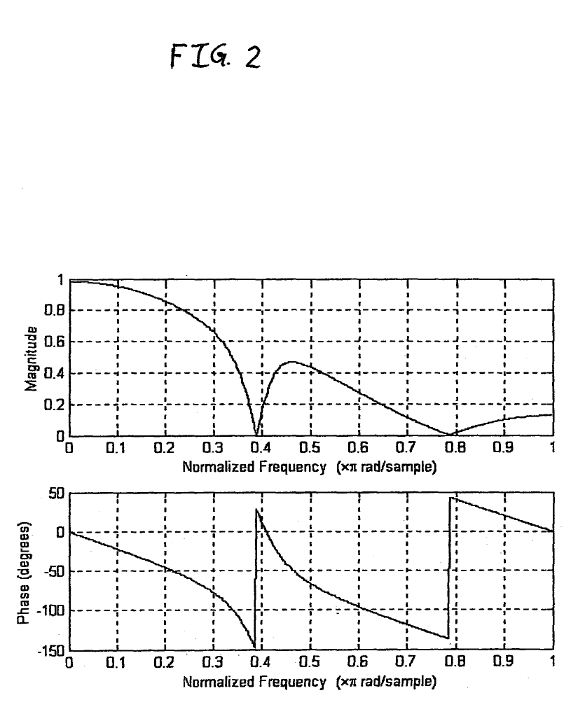

Particularly preferred coefficients for filtering 50 Hz noise from digitized input signal data input into the circuit were found to be: coefficient A, 115/128; coefficient B, 79/128; coefficient C, 9/32; coefficient D, 7/16; coefficient E, 9/16; and coefficient F, ¾. A Z-plane plot for a 50 Hz filter implementing these particularly preferred coefficients is provided in FIG. 1. The frequency response of this filter is shown in FIG. 2.

To filter 60 Hz noise, preferred values for coefficients A-F were in the following ranges:

| coefficient | minimum value | maximum value |
|---|---|---|
| A | about 852/1024 | about 940/1024 |
| B | about 159/1024 | about 177/1024 |
| C | about 244/1024 | about 268/1024 |
| D | about 472/1024 | about 520/1024 |
| E | about 182/1024 | about 202/1024 |
| F | about 730/1024 | about 806/1024 |

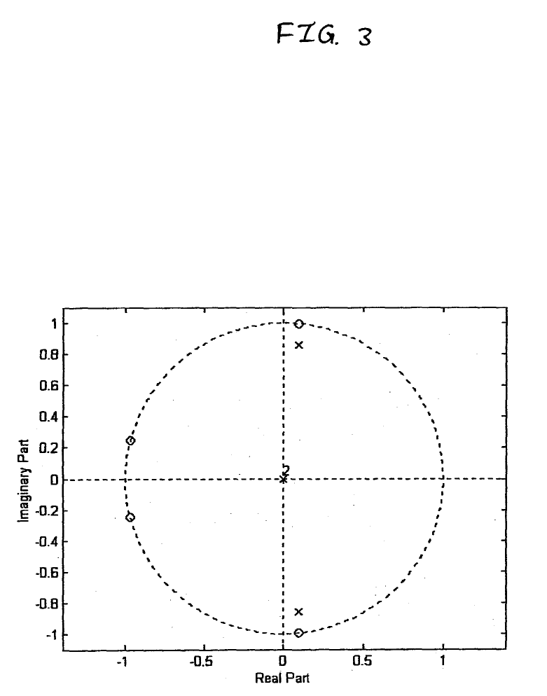

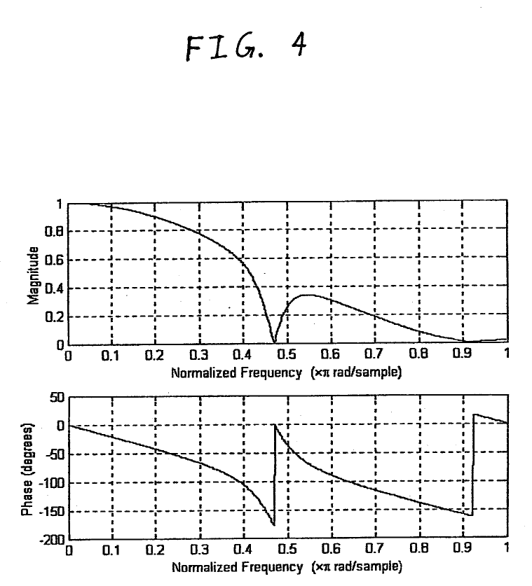

Particularly preferred coefficients for filtering 60 Hz noise from digitized input signal data input into a circuit capable of implementing the transfer function of Eq. (1) were found to be: coefficient A, ⅞; coefficient B, 21/128; coefficient C, ¼; coefficient D, 31/64; coefficient E, 3/16; and coefficient F, ¾. A Z-plane plot for a 60 Hz filter implementing these particularly preferred coefficients is provided in FIG. 3. The frequency response of this filter is shown in FIG. 4.

Numerous alternatives to the transfer function of Eq. (1) and the coefficients listed above may be used in the practice of the invention, and are left to the discretion of the skilled artisan in view of the instant teachings. In designing individual filters to implement different known or later developed transfer functions, any suitable approach can be used. One such approach involves the use of MATLAB® software (The MathWorks, Inc., Natick, Mass.). Using such software, one can select the type of desired filter and the frequency(ies) to be removed from an input signal by specifying poles and zeros. The software derives coefficient approximations. From these approximations, different coefficients (typically within about 5% of the particular coefficient approximation) can be tested. For hardware implementation, fractions can then be developed for the particular coefficients. It is preferred that each fraction be a power of two, with as small a denominator as possible. If floating point calculations are appropriate in the given context, other fractions may be used. As will be appreciated, such calculations can also be performed using software, and software embodying the hardware-based embodiments of the invention can be developed in view of the instant disclosure.

As those in the art will appreciate, because the control logic of a selectable notch filter according to the invention can be embedded in hardware or implemented as software, to conserve energy and avoid the need for floating point arithmetic, it is preferred that the values of coefficients in a coefficient set used in implementing a transfer function for a particular filter of a selectable notch filter circuit each be a fraction, preferably a fraction the denominator of which is a factor of two, or a decimal representation thereof. Most preferred are fractions having a denominator as small as possible but which still allow the corresponding filter to provide the desired response.

The number and diversity of notch filters in a given selectable notch filter circuit will be dictated by the number and diversity of the coefficient sets available to the control logic of the circuit. As few as two different coefficient sets may be available. In contrast, the circuit's control logic may have 3 or more different coefficient sets available or accessible to it. Alternatively, a notch filter circuit may contain two or more sub-circuits, each of which is configured as a notch filter and each of which, for example, implements a different coefficient set, thereby allowing each of the filters to remove a different interfering fundamental frequency (or small range of frequencies) and at least its second harmonic from a digitized signal input into the filter. Also, additional selectable notch filter circuits may also be included in the operational circuitry of a device having an internal electrical signal that may require filtering.

In still other embodiments, two or more different notch filters for the same designated fundamental frequency may be used in a single device. In such cases, different coefficient sets may be available for implementation by the control logic of a single selectable notch filter circuit. Alternatively, the different filters for the same designated fundamental frequency may reside in different selectable notch filter circuits. Such filters may use the same control logic to implement the same transfer function, the difference being that two different coefficient sets are used. In this context, one coefficient set will be understood to differ from the other in so long as the coefficient value for at least one variable common to both sets differs from one set to the next. In an alternative approach, the different circuits may implement different transfer functions.

In a selectable notch filter circuit, one or more of the coefficient sets may be stored in the circuit itself or, alternatively, one or more of them may be accessed from a memory separate from, but operably connected with, the circuit, for instance, in a look-up table stored in an associated memory.

b. Noise Detection.

As described above, a notch filter according to the invention selectively filters a digitized input electrical signal to eliminate electrical interference, or “noise”, that lies within the frequency spectrum of a digitized electrical signal input into the circuit. Noise can result from a variety of causes, including the environment in which it operates, e.g., at home, abroad, and at work. Moreover, electrical interference that can interfere with the operation of sensitive electronics, as may be found in implantable medical devices such as ICDs, may be caused by passing the device near a metal detector, a radio transmitter, a welder, a security surveillance system, a microwave generator, etc. Failure to detect such noise may render the device temporarily inoperative or, perhaps more seriously, cause it to function improperly. Clearly, in the context of implantable medical devices such as ICDs, any inoperability or improper function may be life threatening. Accordingly, unless filtering of suspected noise frequencies is to be continuous, interference existing in electronic signals within the device, particularly those flowing through its sensory portions, should be accurately detected.

As described above, filtering of electrical interference can be continuous, regardless of whether such interference is indeed present in the signal at a given time. Such an approach, while often acceptable, may consume more power than necessary. When power consumption and conservation are important considerations, as is true for implantable medical devices such as ICDs, it may be preferred to perform such filtering only intermittently. For example, in preferred embodiments, because filtering requires computation, to conserve power, filtering is performed intermittently, most preferably only when noise actually exists in the input signal.

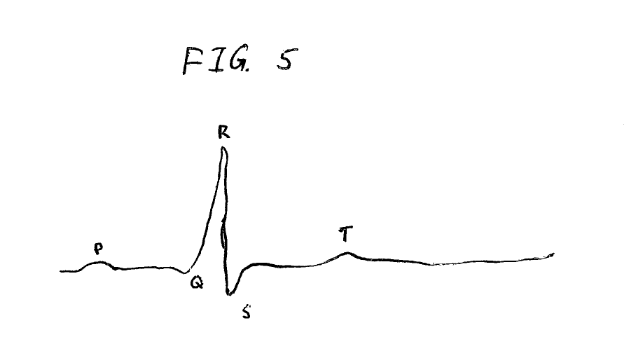

To detect the presence of noise in the input signal, any suitable approach can be employed and adapted as necessary for purposes of the invention. For example, with regard to ICDs, it is desirable to detect noise in the signal emanating from a sensing electrode at a time when no event is expected that corresponds to the physiological parameter being monitored. As a representative example, in the context of a human heart, a sensing electrode typically monitors the heart's electrical activity, which differs during the various phases of a single heart beat. With reference to FIG. 5, a typical normal human heartbeat has several phases. Briefly, the P-wave represents the heart's electrical activity during atrial contraction. The “QRS” complex represents the heart's electrical activity during ventricular contraction (caused by depolarization of ventricular muscle), and the T-wave represents a ventricular repolarization wave (due to ventricle relaxation). Monitoring changes in one or more of heart rate (for example, by determining the time interval between the R-wave peak of successive heart beats) and waveform morphology, amplitude, and frequency content allow abnormal cardiac conditions to be detected.

As is evident from FIG. 5, there are various times during each heartbeat when a there is little to no expected electrical activity to be detected. Any one or more of these different periods during each heartbeat (or, alternatively, intermittently, e.g., at the same time during every third heart beat), an assessment can be made to determine if noise is present and, if so, of what fundamental frequency. If noise is detected, the appropriate notch filter can be selected that corresponds to the detected fundamental frequency of the noise. Thereafter, filtering may be continued for a pre-determined interval (e.g., 30, 60, 90, 120, or more seconds), after which the system resets and begins its noise detection process anew. Alternatively, noise detection can continue uninterrupted, and when noise is no longer detected, filtering of the particular noise frequency(ies) can be halted until such time as noise is again detected.

A preferred noise detection process useful in conjunction with certain embodiments of the invention, for example, with ICDs, simply assesses how many times, if at all, the input signal oscillates through zero amplitude (representing a direct current) or a preset threshold other than zero over a preset interval within a time period of a cardiac cycle when no oscillation in the signal is expected (e.g., the time period that begins after dissipation of an S-wave but before the repolarization of the ventricle begins). If 50 Hz noise is present, the signal would oscillate through zero (or another pre-determined threshold) about every 20 ms (milliseconds). For 60 Hz noise, the period would be about 16.67 ms. Thus, if 50 Hz or 60 Hz noise was present in the signal, during a window of about 25 ms during the refractory period between the end of an S-wave and before a T-wave, if the signal oscillates through zero (or another threshold) three or four times, the noise detection circuit would signal to the selector that noise is present, and of what fundamental frequency (50 Hz in the case when three threshold-crossings are detected, and 60 Hz when four threshold-crossings are detected). Any suitable time window may be employed, although shorter windows are preferred. Likewise, the results of a series of discrete time windows in a particular period may be used in assessing whether noise is present. For example, did at least two or more windows within a period the length of 3-4 windows result in an indication that noise was present? If so, the result of the next succeeding window in the period could be used as the determinant as to whether noise was present.

Of course, as those in the art will appreciate, what constitutes an instance of when the input signal crosses through, or exceeds, “zero” or any other pre-determined threshold is a matter left to the artisan's choice. Here, “zero” refers to a signal the amplitude of which does not exceed a preset threshold. Being below the threshold means that the signal oscillation is not attributable to noise external to the device or that it is insufficient to interfere with proper operation of the device if not filtered from the input signal. If an oscillation is detected that has an amplitude below the threshold, it will not be used in the noise detection process. A signal whose amplitude meets or exceeds the threshold in such cases, however, will be used. Setting the particular threshold will be influenced by many factors, including the type of device, its operational environment, the presence of shielding, the device's power supply, etc. Even so, in a preferred embodiment, it is desirable that the threshold be about 5% that of the average amplitude of an R-wave of a healthy subject.

Those in the art will also appreciate that many variations on the above theme exist, that the foregoing noise detection process is merely representative, and that any process yielding the same output (i.e., whether noise amenable to filtering by the selectable circuit is present, and if so, its fundamental frequency) may be adapted and used in a device according to the invention.

c. Filter Selection.

As described herein, the instant selectable notch filter circuits allow different interfering fundamental frequencies (and at least their second harmonic frequency) to be removed from an input electrical signal in a device incorporating the circuit. Accordingly, such circuits can be used to filter noise of specific frequencies from input signals. Once noise of a filterable fundamental frequency (i.e., corresponding to a fundamental frequency that can be filtered by the circuit) is detected, it can be filtered. If, at the time noise is detected, the filter is either not then being used, or, alternatively, is set to filter a different fundamental frequency and second harmonic thereof from the input signal, a selector can reconfigure the filter to filter the then-detected noise. In some embodiments, filtering will not occur unless the strength of the interfering frequency exceeds a minimum (typically preset, or pre-programmed) threshold, the selector can direct the filter circuit to filter the particular frequency or, depending on the filter configuration, select which of several different filter outputs to use. As those in the art will appreciate, if the gain of the input signal is to be amplified prior to analysis, in embodiments where the strength of an interfering frequency must exceed a threshold, the threshold should be set at a level low enough such that post-amplification any residual interference in the signal should not adversely impact analysis of the input signal.

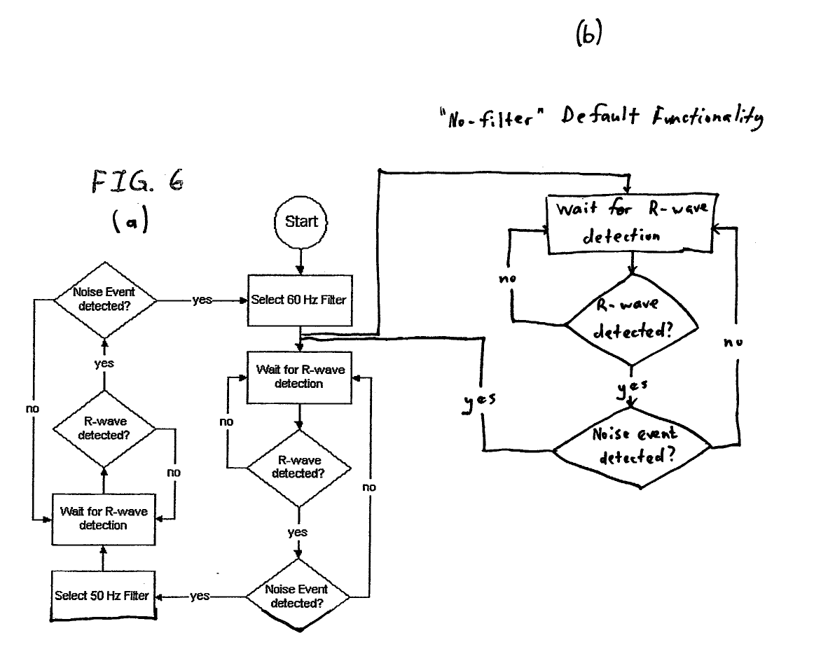

FIG. 6(a) depicts a representative method for selecting which of two notch filters in a selectable notch filter circuit should be selected in a system that uses continuous monitoring. In the embodiment represented part (a) of the figure, the selectable notch filter circuit can deploy either of two notch filters, a 60 Hz notch filter or a 50 Hz notch filter. In this embodiment, the 60 Hz notch filter is selected as a default filter, perhaps because a device incorporating the circuit is being implanted in a patient who resides in the United States, and the input signal is monitored for the presence of 60 Hz noise. Here, noise detection occurs after R-wave detection, preferably in a quiescent period in the input signal (i.e., a period when no electrical activity from the heart is expected to be detected, for example, after the R-wave subsides but before the T-wave is initiated. If noise is not detected, 60 Hz filtering continues. If noise, however, is detected, the selector reconfigures the circuit logic to filter 50 Hz noise. Noise detection continues after subsequent R-waves until such time as noise is again detected in the input signal, at which time the selector then reconfigures the circuit logic to filter 60 Hz noise. Noise detection can occur after each R-wave (i.e., continuously), but is preferably performed intermittently. For example, noise detection could be performed after every ith R-wave, where i is an integer, e.g., 10, 50, 100, or more. Alternatively, the noise detection routine could be run according to a pre-determined time interval, for example, every 10, 30, 60, 120, or more seconds. Also, various combinations of such routines could be run, depending on the conditions encountered by the device. For instance, immediately after a filter switch, it may be desirable to more frequently monitor for the presence of noise. If no noise is detected for a preset period, as may occur after a patient takes up residence in a location where the line frequency is 50 Hz after leaving a location where the line frequency was 60 Hz (e.g., as would occur upon traveling to Europe from the United States), noise detection may be preformed less frequently to conserve energy.

Part (b) of FIG. 6 depicts a preferred, yet optional, aspect in the decision tree that can be implemented by a circuit according to the invention. In embodiments of this sort, the default setting is a “no-filter,” and hence energy conserving, function. Unless a noise event is detected in the signal, no filtering is performed. If noise is detected in the input signal, filtering is then performed.

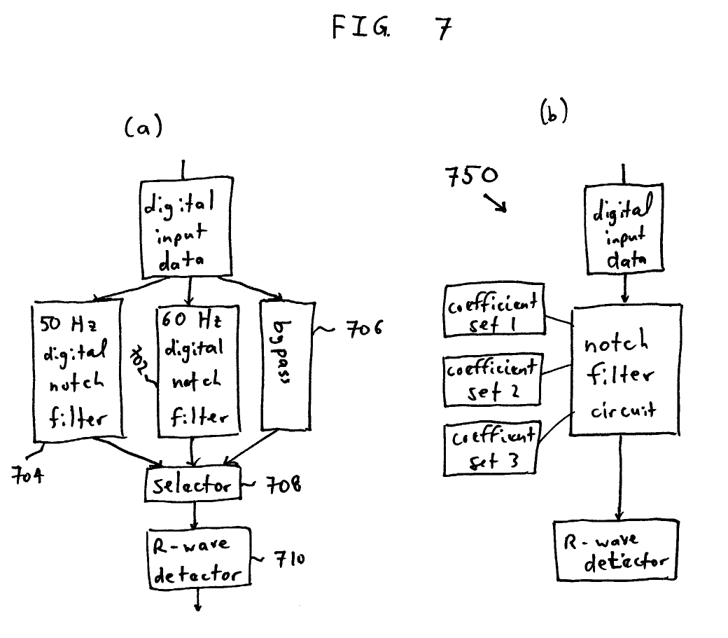

FIG. 7 depicts a selectable notch filter circuit in which either of two notch filter capabilities (60 Hz notch filtering (702) and 50 Hz notch filtering (704)) can be selected at a given time. Alternatively, the filtering capability can be bypassed (706). Which filter output, if any, to be used at a given time is determined by the selector (708). After filtering, if any, the input signal passes out of the selectable notch filter circuit for analysis by R-wave detector (710) or other wave analysis circuitry. In the event a noise event was detected, an “interrupt” signal would be sent to the central processing unit to ensure that filtration of the detected noise from the signal thereafter input into the wave detector is performed for at least some minimum number of cycles.

3. Devices and Applications.

Selectable notch filter circuits according to the invention may be included in the operational circuitry of any electronic device through which one or more data-carrying electric signals flow. When incorporated, one of the several notch filters embodied in the circuit may be deployed to remove electrical interference having the designated fundamental frequency of the selected filter. Because the circuit embodies the capability to implement any of a plurality of notch filters each specific for a different fundamental frequency and a second harmonic thereof, interference of different fundamental frequencies can, if desired, effectively be filtered from the data-carrying electric signals. Devices in which such circuits will find application include telecommunications devices (e.g., mobile and cellular telephones) and personal digital assistants and other portable computing devices. Perhaps an even more important class of devices for deployment of the invention's circuits is the class of implantable medical devices. Such devices include implantable drug pumps, artificial hearts and left ventricle assist devices, pacemakers, and implantable defibrillators, including ICDs. While the following discussion will focus on ICDs, these teachings may be readily adapted to any other class of electronic device.

ICDs are used to counter arrhythmic heart conditions, including arrhythmias of the atria and ventricles. Arrhythmias include bradycardia and tachycardia. An arrhythmia is any variation from the normal rhythm of the heartbeat; it may be an abnormality of either the rate, regularity, or site of impulse origin or the sequence of activation. The term encompasses abnormal regular and irregular rhythms as well as loss of rhythm. Bradycardia is an abnormally slow or irregular heart rhythm (usually less than 60 beats per minute). It causes symptoms such as dizziness, fainting, extreme fatigue, and shortness of breath due to insufficient oxygenation of the body's tissues caused by less than adequate blood flow from the heart.

Tachyarrhythmia is an abnormally fast heart rhythm (usually 100-400 beats per minute) in either the atria (atrial tachyarrhythmia) or ventricles. There are two types of atrial tachyarrhythmia, atrial fibrillation (AF) and atrial flutter. Atrial fibrillation (AF) occurs when the right and left atria quiver (typically at the rate of about 300-600 bpm) instead of beating effectively to pump blood into the ventricles. As a result, blood may pool and clot in the atria. If a clot dislodges, and advances to the brain, it can cause a stroke. Atrial flutter is a rapid, regular heartbeat wherein the atria still pump blood at the rate of about 250-350 bpm, causing the ventricles to pump at about half that rate, which is not as efficient as during a normal sinus rhythm.

Ventricular fibrillation (VF) is a specific type of tachyarrhythmia, and refers to a very fast, irregular heart rhythm in the right and left ventricles. During VF, the heart quivers and pumps little or no blood to the body. VF causes loss of consciousness in seconds, and is fatal if not immediately treated and a more normal heart rhythm restored. Ventricular tachycardia is a less severe ventricular tachyarrhythmia than VF, and does not result in a complete of loss of blood pumping action.

The current standard of care for treating arrhythmias includes implanting cardioverters/defibrillators (with or without pacing capability) in patients diagnosed with these chronic disorders. Such devices are used to counter arrhythmic heart conditions by stimulating the heart with electrical impulses or shocks of a magnitude substantially greater than pulses used in cardiac pacing.

Conventional cardioversion/defibrillation systems typically include an implanted cardioverter/defibrillator, one or more body-implantable, electrically insulated leads containing one or more electrodes that are connected to cardioverter/defibrillator, and programming mechanism that can be used to remotely program the electronics of the cardioverter/defibrillator. Cardioverter/defibrillators generally consist of a hermetically sealed container housing the device's electronics (also referred to herein as “operational circuitry”), battery supply, and capacitors. Conventional ICD electrodes can be in the form of patches applied directly to epicardial tissue (see U.S. Pat. Nos. 4,567,900; 5,618,287; and 5,476,503), or, more commonly, are “intravascular” or “transvenous” electrodes disposed in the distal regions of small cylindrical insulated catheters surgically implanted in one or more endocardial areas of the heart through the superior vena cava. See U.S. Pat. Nos. 4,603,705; 4,693,253; 4,944,300; and 5,105,810. The implantable cardioverter/defibrillator and lead(s) of such systems are referred to herein as implantable cardioverter/defibrillators, or “ICDs”.

Currently marketed ICDs are small enough to be implanted in the pectoral region. Advances in circuit design has also led to ICDs where the housing forms a subcutaneous electrode. See U.S. Pat. Nos. 5,133,353; 5,261,400; 5,620,477; and 5,658,321. As ICD therapy becomes more prophylactic in nature and is used in progressively less ill individuals, especially children at risk of cardiac arrest, the requirement of ICD therapy to use intravenous catheters and transvenous leads has become an impediment, as most individuals will begin to develop complications related to lead system malfunction sometime within the devices' 5-10 year operational lifetime, and since chronic transvenous lead reimplantation and removal can damage major cardiovascular venous systems and the tricuspid valve, as well as result in life threatening perforations of the great vessels and heart, especially in patients with life expectancies of more than about five years and/or who are growing (i.e., children).

To overcome the deficiencies of currently available ICDs, recently two new ICD classes have been developed. These classes are subcutaneous ICDs (S-ICDs), which are implanted subcutaneously in the area of a patient's ribcage but still comprise one or more leads connected to the cardioverter/defibrillator portion of the device, and unitary S-ICDs (US-ICDs), which have electrodes integrated into the housing (collectively, these devices are referred to as S-ICDs). These devices include a housing that conforms to a patient's ribcage when subcutaneously positioned (for example, in an intercostal space), one or more sensing and treatment electrodes disposed in the housing such that proper electrode positioning is achieved upon implantation, electrical circuitry located within the housing for monitoring electrical activity of the patient's heart to sense if an abnormal cardiac rhythm occurs, in which event the device administers an appropriate electrical stimulus, or series of stimuli to treat the condition and restore a normal sinus rhythm, and a long-lasting battery set sufficient to power the device's sensing and treatment circuitry. Such devices are thoroughly described in U.S. patent applications in published U.S. patent applications having the following publication numbers: 20020120299A1; 20020107559A1; 20020107549A1; 20020107548A1; 20020107547A1; 20020107546A1; 20020107545A1; 20020107544A1; 20020103510A1; 20020091414A1; 20020072773A1; 20020068958A1; 20020052636A1; 20020049476A1; 20020049475A1; 20020042634A1; 20020042630A1; 20020042629A1; 20020035381A1; 20020035380A1; 20020035378A1; 20020035377A1; and 20020035376A1.

As those in the art will appreciate, the operational circuitry of ICDs and S-ICDs (as well as other electronic devices sensitive to electrical interference) can be improved by inclusion of one or more selectable notch filter circuits according to the invention. This can be accomplished, for example, by inclusion of one more circuits of the invention into the device's operational circuitry during its design phase. A device according to the invention may also include a diagnostic capability to determine if one or more of the filters of selectable notch filter circuit are functioning properly and, if not, taking a corrective action, e.g., logging such failure for later retrieval, activating an alarm signal, etc.

As with other ICDs, S-ICDs according to the invention contain circuitry to monitor cardiac rhythms. If an abnormal rhythm is detected, the device initiates charging of its capacitor. If the abnormal rhythm is confirmed, the cardioversion/defibrillation energy is delivered via one or more electrodes. In the case of S-ICDs, the treatment energy is typically delivered through the active surface of the device's housing and a subcutaneous electrode. Examples of such systems are described in U.S. Pat. Nos. 4,693,253 and 5,105,810.

An ICD, including an S-ICD, according to the invention preferably can provide cardioversion/defibrillation energy in different types of waveforms, as appropriate. Any waveform useful in treating the particular abnormal rhythm can be used. Representative waveforms include monophasic, biphasic, multiphasic, or alternative waveforms. For instance, a 100 uF biphasic waveform of approximately 10-20 ms total duration and with the initial phase containing approximately ⅔ of the energy can be used.

In addition to providing cardioversion/defibrillation energy, an ICD according to the invention can also provide transthoracic cardiac pacing capability. This can be accomplished by including circuitry for monitoring the heart for bradycardia and/or tachycardia rhythms in the device. If a bradycardia or tachycardia rhythm is detected in a patient, the circuitry can then deliver appropriate pacing energy at appropriate intervals through, for example, active surface and subcutaneous electrodes. In some embodiments, pacing stimuli are biphasic and similar in pulse amplitude to those used for conventional transthoracic pacing.

Pacing capability can also be used to provide low amplitude shocks on the T-wave for induction of ventricular fibrillation for testing S-ICD performance in treating VF (see U.S. Pat. No. 5,129,392). Pacing circuitry can also be used to rapidly induce ventricular fibrillation or ventricular tachycardia. VF can also be induced by providing a continuous low voltage, i.e., about 3 volts, across the heart during the entire cardiac cycle.

ICDs according to the invention can also be engineered to detect and treat atrial rhythm disorders, including atrial fibrillation. See Olson, et al. (1986), Computers in Cardiology, pp. 167-170. In such cases, the ICD will have two or more electrodes that provide the ability to record the P-wave of the electrocardiogram as well as QRS waves. These electrodes may be the same or different from those used to monitor the ventricles. One can detect the onset and offset of atrial fibrillation using any suitable method, including R-R cycle length instability detection algorithms and algorithms to detect changes in P-wave morphology. Once AF has been detected, the operational circuitry can then provide appropriate therapy, e.g., QRS synchronized atrial defibrillation/cardioversion using the same shock energy and waveform characteristics used for ventricular defibrillation/cardioversion.

In preferred embodiments, the sensing circuitry of an ICD will utilize electronic signals generated from the heart primarily to detect QRS waves. For ventricular tachycardia or fibrillation detection, the circuitry preferably uses a rate detection algorithm to trigger capacitor charging once the ventricular rate exceeds some predetermined threshold for a fixed period of time. For example, if the ventricular rate exceeds 240 bpm on average for more than 4 seconds, the capacitor will be charged. A confirmatory rhythm check is then performed to ensure that the rate persists for at least about another one second before discharge. If the confirmatory check reveals that the abnormal has not persisted, a termination algorithm could be instituted to drain the charged capacitor charge to an internal resistor. Now known or later developed detection, confirmation, and termination algorithms such as these can be readily adapted for use with devices of the instant invention.

With regard to ICDs of the invention, the housing is a hermetically sealed shell that encases the operational circuitry and battery supply for the device. The primary function of the housing is to provide a protective barrier between the electrical components and circuitry held within its confines and the surrounding environment. Accordingly, the housing should possess sufficient hardness to protect its contents. Materials possessing this hardness include numerous suitable biocompatible materials such as medical grade plastics, ceramics (e.g., zirconium ceramics and aluminum-based ceramics), metals (e.g., stainless steel and titanium), and alloys (e.g., stainless steel alloys and titanium alloys such as nickel titanium). Although the materials possessing such hardnesses are generally rigid, in particular embodiments (e.g., S-ICDs), it is desirable to utilize materials that are pliable or compliant, including those capable of partially yielding without fracturing. Examples of compliant materials include polyurethanes, polyamides, polyetheretherketones (PEEK), polyether block amides (PEBA), polytetrafluoroethylene (PTFE), polyethylene, silicones, and mixtures thereof. Of course, device housing may comprise combinations of these and other materials as well. For example, a nonconductive polymeric coating, such as parylene, may be selectively applied over portions of a titanium alloy housing to provide only specific surface areas that can receive signals and/or apply therapy. In preferred embodiments, the housing has a volume of less than about 60 cc and a weight of less than about 100 g for long term wearability, especially in children. Examples of small ICD housings are disclosed in U.S. Pat. Nos. 5,597,956 and 5,405,363. The housing and lead of an ICD or S-ICD can also use fractal or wrinkled surfaces to increase surface area to improve defibrillation capability.

A device housing may include one or more apertures, sensors, electrodes, appendages, or combinations thereof. Apertures in the housing are generally in the form of connection ports for coupling ancillary devices (e.g., a lead electrode for sensing, shocking, and pacing) to the operational circuitry in the housing.

Any sensor capable of receiving physiological information (i.e., a “sensing” or “diagnostic” electrode) and/or emitting an energy (i.e., a “therapy” or “shocking” electrode) may be situated in the housing so that its electrically conductive surface is positioned at the surface, or in a recess at the surface, of the housing. For example, a sensor may be located on the housing to monitor a patient's blood glucose level, respiration, blood oxygen content, and blood pressure, and/or cardiac output. Sensors may also be located in leads that are electrically coupled to the operational circuitry encased within the housing. Sensing and/or therapy electrodes disposed in leads may perform many of the functions defined by the operational circuitry's programming. In many cases, they are the vehicles that actually receive the signals being monitored and/or emit the energy required to pace, shock, or otherwise stimulate the patient's heart. Multiple, task-specific electrodes (i.e., perform a single function) may be used, as can one or more electrodes that perform both monitoring and therapy (i.e., shocking) functions.

For therapy, the ICDs of the present invention provide an energy (measured in a suitable energy unit, e.g., electric field strength (V/cm), current density (A/cm²), or voltage gradient) to a patient's heart. Such devices will generally use voltages in the range of about 700 V to about 3150 V, requiring energies of approximately 40 J to 210 J. Energy requirements will vary, however, depending upon the form of treatment, the proximity of the device to the patient's heart, the relative position of the therapy electrodes to each other, the nature of the patient's underlying heart disease, the specific cardiac disorder being treated, and the ability to overcome diversion of the device's electrical output into other thoracic tissues. Ideally, energy emitted from the device will be directed into the patient's anterior mediastinum, through the majority of the heart, and out to the coupled lead electrode positioned in the posterior, posterolateral, and/or lateral thoracic locations. Furthermore, it is desirable that the device be capable of delivering this directed energy, as a general rule, at an adequate effective field strength of about 3-5 V/cm to approximately 90 percent of a patient's ventricular myocardium using a biphasic waveform.

When delivering therapy, the devices provide energy with a sufficient pulse width to achieve the desired result, e.g., cardioversion or defibrillation. Preferably, the pulse widths are approximately one millisecond to approximately 40 milliseconds. For pacing, the devices also provide an appropriate level of pacing current, preferably about one milliamp to approximately 250 milliamps.

Any suitable electrode can be used in a device according to the invention. Preferred electrodes are subcutaneous electrodes. Preferably, the electrode lead has silicone or polyurethane insulation, with the electrode being connected to the housing via a suitable connection port. Electrodes include composite electrodes having multiple electrodes attached to the housing via a common lead. It is preferred that electrodes connected via leads be anchorable into soft tissue such that the electrode does not dislodge after implantation.

In ICDs and S-ICDs, a plurality of electrodes, for example, three, may be present. In some such devices, a composite subcutaneous electrode is used, and comprises a coil electrode for delivering high voltage cardioversion/defibrillation energy across the heart and two insulated, proximally placed sense electrodes spaced sufficiently (e.g., about 1-10 cm, with 4 cm being preferred) to allow for good QRS wave detection. See U.S. Pat. No. 5,534,022. As those in the art will appreciate, any suitable electrode configuration for delivery of cardioversion and defibrillation energy and sensing can be used in the context of this invention. For example, configurations having only one sensing electrode, either proximal or distal to a cardioversion/defibrillation electrode, which can serve both as a sensing electrode and a cardioversion/defibrillation electrode.

Sensing of cardiac waveforms (e.g., QRS waves) and/or transthoracic impedance can be carried out via sense electrodes on the housing of an S-ICD or in combination with a cardioversion/defibrillation electrode and/or one or more subcutaneous lead sensing electrodes. Placing sensing electrodes on the housing eliminates the need for sensing electrodes on the subcutaneous electrode. It is also contemplated that a subcutaneous electrode can be provided with at least one sense electrode, the housing with at least one sense electrode, and if multiple sense electrodes are used on either the subcutaneous electrode and/or the housing, that the best QRS wave detection combination be identified when the S-ICD is implanted. In this way, the best sensing electrode combination can be selected from all the existing sensing possibilities. For example, in embodiments having four sensing electrodes, e.g., two on the subcutaneous lead and two on the housing, the S-ICD may have a programmable feature that allows it to be adapted to changes in the patient physiology and size (in the case of children) over time. Programming can be accomplished via any suitable approach, for example, using physical switches on the device housing, or preferably, via the use of a programming wand or other wireless connection.

The optimal subcutaneous placement of an S-ICD is in a subcutaneous space developed during the implantation process, and will vary depending upon the exact design of the particular device and the anatomy of the particular patient. In many adult patients, this space will be located in the left mid-clavicular line approximately at the level of the inframammary crease at approximately the 5th rib when the device uses a subcutaneous electrode. In children, a representative placement for an S-ICD has the device housing located in the left posterior axillary line approximately lateral to the tip of the inferior portion of the scapula. When such devices employ a subcutaneous sensing and therapy electrode attached to the main body of the device, the lead of the subcutaneous electrode typically traverses a subcutaneous path around the thorax terminating with its distal electrode at the posterior axillary line, ideally just lateral to the left scapula in adults. In children, the distal electrode end is placed at or near the anterior precordial region, ideally in the inframammary crease. Such placements provide a reasonably good pathway for current delivery between a cardioversion/defibrillation electrode in the device housing and the subcutaneous electrode to the majority of the ventricular myocardium.

All patents and patent applications, publications, scientific articles, and other referenced materials mentioned in this specification are indicative of the levels of skill of those skilled in the art to which the invention pertains, and each of which is hereby incorporated by reference to the same extent as if each reference had been incorporated by reference in its entirety individually. Applicants reserve the right to physically incorporate into this specification any and all materials and information from any such patents and patent applications, publications, scientific articles, electronically available information, and other referenced materials or documents.

The specific circuits, algorithms, transfer functions, machines, and methods described in this specification are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Also, the terms “comprising”, “including”, “containing”, etc. are to be read expansively and without limitation. It must be noted that as used herein and in the appended claims, the singular forms “a”, “an”, and “the” include plural reference unless the context clearly dictates otherwise.

The terms and expressions that have been employed are used as terms of description and not of limitation, and there is no intent in the use of such terms and expressions to exclude any now-existing or later-developed equivalent of the features shown and described or portions thereof, but it is recognized that various modifications are possible within the scope of the invention as claimed. Thus, it will be understood that although the present invention has been specifically disclosed by preferred embodiments and optional features, modification and/or variation of the disclosed elements may be resorted to by those skilled in the art, and that such modifications and variations are within the scope of the invention as claimed.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A circuit comprising hardware configured to serve at a given time as a selected one of a plurality of notch filters each of which is specific for filtering in real time from digitized input signal data input into the circuit a different designated fundamental frequency and a second harmonic thereof, wherein embedded in the hardware is control logic that controls filtering of a sampled designated fundamental frequency by using a coefficient set specific for the sampled designated fundamental frequency selected from among a plurality of coefficient sets each specific for a different designated fundamental frequency, and wherein each of the notch filters operates on digitized input signal data sampled at a substantially equivalent sampling rate.

2. A circuit according to claim 1 wherein the designated fundamental frequency is about 60 Hz.

3. A circuit according to claim 1 wherein the designated fundamental frequency is about 50 Hz.

4. A circuit according to claim 1 wherein one of the notch filters is specific for filtering a designated fundamental frequency of about 60 Hz and another of the notch filters is specific for filtering a designated fundamental frequency of about 50 Hz.

5. A circuit according to claim 1 wherein the sampling rate is greater than about 240 Hz.

6. A circuit according to claim 1 wherein the sampling rate is between about 240 Hz and about 2,400 Hz.

7. A circuit according to claim 1 wherein the sampling rate is greater than about 240 Hz and less than about 600 Hz.

8. A circuit according to claim 7 wherein the sampling rate is about 256 Hz.

9. A circuit according to claim 2 wherein the coefficient set comprises coefficients A, B, C, D, E, and F for solving a transfer function defined by Eq. (1), as follows:
$$H ( z ) = \frac{( A - B \cdot {z}^{- 1} + A \cdot {z}^{- 2} ) \cdot ( C + D \cdot {z}^{- 1} + C \cdot {z}^{- 2} )}{1 - E \cdot {z}^{- 1} + F \cdot {z}^{- 2}} \qquad Eq . ( 1 )$$
wherein each of coefficients A-F is a fraction or a decimal representation thereof selected from the following:

| coefficient | minimum value | maximum value |
|---|---|---|
| A | about 852/1024 | about 940/1024 |
| B | about 159/1024 | about 177/1024 |
| C | about 244/1024 | about 268/1024 |
| D | about 472/1024 | about 520/1024 |
| E | about 182/1024 | about 202/1024 |
| F | about 730/1024 | about 806/1024 |

10. A circuit according to claim 9 wherein coefficient A is about ⅞ or a decimal representation thereof, coefficient B is about 21/128 or a decimal representation thereof, coefficient C is about ¼ or a decimal representation thereof, coefficient D is about 31/64 or a decimal representation thereof, coefficient E is about 3/16 or a decimal representation thereof, and coefficient F is about ¾ or a decimal representation thereof.

11. A circuit according to claim 3 wherein the coefficient set comprises coefficients A, B, C, D, E, and F for solving a transfer function defined by Eq. (1), as follows:
$$H ( z ) = \frac{( A - B \cdot {z}^{- 1} + A \cdot {z}^{- 2} ) \cdot ( C + D \cdot {z}^{- 1} + C \cdot {z}^{- 2} )}{1 - E \cdot {z}^{- 1} + F \cdot {z}^{- 2}} \qquad Eq . ( 1 )$$
wherein each of coefficients A-F is a fraction or a decimal representation thereof selected from the following:

| coefficient | minimum value | maximum value |
|---|---|---|
| A | about 874/1024 | about 966/1024 |
| B | about 600/1024 | about 664/1024 |
| C | about 274/1024 | about 302/1024 |
| D | about 426/1024 | about 472/1024 |
| E | about 548/1024 | about 604/1024 |
| F | about 730/1024 | about 806/1024 |

12. A circuit according to claim 11 wherein coefficient A is about 115/128 or a decimal representation thereof, coefficient B is about 79/128 or a decimal representation thereof, coefficient C is about 9/32 or a decimal representation thereof, coefficient D is about 7/16 or a decimal representation thereof, coefficient E is about 9/16 or a decimal representation thereof, and coefficient F is about ¾ or a decimal representation thereof.

13. A circuit according to claim 1 wherein the control logic implements a transfer function that utilizes a floating point calculation.

14. A circuit according to claim 1 wherein the hardware further comprises the coefficient set specific for the sampled designated fundamental frequency embedded therein.

15. A circuit according to claim 1 wherein the control logic is capable of accessing at least one of the coefficient sets from a memory operably associated with but separate from the circuit.

16. A selectable notch filter circuit for an implantable medical device, comprising:
a. an input for a digitized electrical signal and an output for the digitized electrical signal;
b. disposed between the input and output, hardware configured to serve at a given time as a selected one of a plurality of notch filters each of which is specific for filtering in real time from the digitized electrical signal input into the circuit a different designated noise fundamental frequency and a second harmonic thereof, wherein embedded in the hardware is control logic that controls filtering of a sampled designated noise fundamental frequency by using a coefficient set specific for the sampled designated noise fundamental frequency selected from among a plurality of coefficient sets each specific for a different designated noise fundamental frequency, and wherein each of the notch filters operates on digitized input signal data sampled at a substantially equivalent sampling rate; and
c. a selector that selects which of the plurality of coefficient sets to be utilized at a given time by the control logic.

17. A selectable notch filter circuit according to claim 16 further comprising a filter bypass for the digitized electrical signal input into the circuit.

18. A selectable notch filter circuit according to claim 17 wherein the selector is capable of selecting the filter bypass.

19. A selectable notch filter circuit according to claim 16 that is operatively connected with circuitry for sensing a physiological parameter of a patient by analysis of the digitized electrical signal.

20. A selectable notch filter circuit according to claim 19 that is operatively associated with circuitry for administering a therapy to the patient if the analysis of the digitized electrical signal is indicative of an abnormal condition.

21. An implantable medical device the circuitry of which contains a selectable notch filter circuit according to claim 16.

22. An implantable medical device the circuitry of which contains a plurality of selectable notch filter circuits each of which is a selectable notch filter circuit according to claim 16.

23. An implantable medical device according to claim 21 for sensing a physiological parameter of a patient.

24. An implantable medical device according to claim 23 that further is capable of administering a therapy to the patient if an analysis of the sensed physiological parameter is indicative of an abnormal condition for sensing a physiological parameter of a patient.

25. An implantable medical device according to claim 23 wherein the physiological parameter is a heart beat.

26. An implantable medical device according to claim 24 wherein the abnormal condition is an arrhythmia.

27. An implantable medical device according to claim 26 wherein the arrhythmia is selected from the group consisting of bradycardia and tachyarrhythmia.

28. An implantable medical device according to claim 27 that is an implantable cardioverter-defibrillator.

29. An implantable cardioverter-defibrillator according to claim 27 that is implanted subcutaneously in a patient.

30. An implantable cardioverter-defibrillator according to claim 29 that does not make physical contact with heart tissue after implantation in the patient.

31. A heart-specific sensing system for an implantable medical device, comprising:
a. an electrode for sensing electrical signals within a patient's body;
b. an analog-to-digital converter for converting a sensed electrical signal to a digitized electrical signal;
c. a selectable notch filter circuit according to claim 16; and
d. a detector to detect whether the digitized electrical signal contains a heart-specific component.

32. A heart-specific sensing system according to claim 31 wherein the detector is an R-wave detector.

33. An implantable cardioverter-defibrillator, comprising:
a. a sense electrode for sensing electrical signals within a patient's body;
b. an analog-to-digital converter for converting a sensed electrical signal to a digitized electrical signal;
c. a selectable notch filter circuit according to claim 16;
d. a detector to detect whether the digitized electrical signal contains a heart-specific component;
e. a therapy electrode for delivering a therapeutic electrical stimulus to the patient's heart when the detector detects in the digitized electrical signal a heart-specific component that is indicative of an abnormal heart rhythm;
f. a power supply; and
g. a housing that houses at least foregoing parts (c), (d), and (f).

34. An implantable cardioverter-defibrillator according to claim 33 that is a subcutaneous unitary cardioverter-defibrillator.

35. A method for filtering an external noise component from a digitized electrical signal in an implantable medical device, comprising:
a. obtaining a digitized electrical signal; and
b. passing the digitized electrical signal through a selectable notch filter circuit according to claim 16.

36. A method according to claim 35 wherein the notch filter initially selected is a default setting.

37. A method according to claim 36 wherein the default setting corresponds to a fundamental frequency of a local AC power supply or a no-filtering function.

38. A method according to claim 37 wherein the fundamental frequency of the local AC power supply is 60 Hz.

39. A method according to claim 37 wherein the fundamental frequency of the local AC power supply is 50 Hz.

40. A method according to claim 35 that is performed intermittently.

41. A method for sensing a physiological parameter of a patient, comprising using an implantable medical device according to claim 23 for sensing the physiological parameter.

42. A method for delivering a therapy to a patient, comprising using an implantable medical device according to claim 24 to deliver the therapy when the sensed physiological parameter indicates that an abnormal condition exists.
